(19) United States
(12) Patent Application Publication
(10) Pub. No.: US 2009/0023960 A1
(43) Pub. Date: Jan. 22, 2009

(54) System for creating dimethyl ether

(76) Inventor: Masood OTAROD, Clarks Summit, PA (US)

Correspondence Address: ZALE Patent Law, 434 Lackawanna Ave., Suite 200, Scranton, PA, 18503-2053, US

(21) Appl. No.: 11/903,448

(22) Filed: Sep. 22, 2007

Related U.S. Application Data
(60) Provisional application No. 60/846,705, filed on Sep. 22, 2006.

(51) Int. Cl. C07C 41/09

(52) U.S. Cl. 568/698

(57) ABSTRACT

The present invention includes an improved system for creating dimethyl ether from gases is described. The process includes introducing hydrogen and carbon monoxide or carbon dioxide into a reaction chamber filled with beads covered with a catalyst. The catalyst includes palladium and one of aluminum, copper, zinc, or mixtures of these which includes an acid side of the molecule. The acid side of the molecule dehydrates methanol, reducing the concentration of this unwanted byproduct and increases the production of diethyl ether. The reactor employs very high temperatures, a defined pressure and defined flow rates to optimize the amount of diethyl ether produced.

### CROSS REFERENCE TO RELATED APPLICATIONS

This application claims priority from U.S. Provisional Patent application 60/846,705 filed Sep. 22, 2006 by the same inventor, Dr. Masood Otarod. The parent Provisional Patent application is incorporated by reference as if set forth in its entirely herein.

### BACKGROUND OF THE INVENTION

1. Field of the Invention

The present invention relates to a system for creating dimethyl ether, and more specifically to a system for creating-methyl ether in a more efficient manner.

2. Discussion of Related Art

Dimethyl ether has the consistency and combustion properties to become a substitute for petroleum products. It is important since it can replace diesel fuels for vehicles and heating oil.

Dimethyl ether has been manufactured in the past from carbon monoxide, hydrogen, carbon dioxide and other gases as described in the paper “In Situ FTIR Spectroscopy And Multiple Isotope Transient Tracing Of The Mechanisms Of Dimethyl Ether Formation From Syngas On Alumina Supported Palladium Catalysts” by the inventor, Dr. Masood Otarod (the 2006 Otarod Paper”), hereby incorporated by reference as if set forth in its entirety herein. Several prior attempts are described in the 2006 Otarod Paper in the section marked “Overview”, however they cannot be optimized since the exact chemical mechanism is not known. Optimization is important since it determines the cost of the fuels created from this process. If the fuels created are significantly more costly than petroleum products, it defeats the goal of creating a substitute.

Currently, there is a need for an optimized system for creating an economical petroleum substitute.

### OBJECTS OF THE INVENTION

It is an object of the present invention to provide a system which creates a petroleum substitute which is less costly.

It is another object of the present invention to provide a system for creating a petroleum substitute which is cleaner burning.

It is another object of the present invention to provide a system which creates a petroleum substitute from readily available resources.

It is another object of the present invention to provide a system which creates a petroleum substitute which can be easily handled.

It is another object of the present invention to provide a system which creates an aerosol substitute from readily available resources.

It is another object of the present invention to provide a system which creates an aerosol substitute which can be easily handled.

### BRIEF DESCRIPTION OF THE DRAWINGS

A complete understanding of the present invention may be obtained by reference to the accompanying drawings, when considered in conjunction with the subsequent detailed description, in which:

FIG. 1 is a flow system used in determining the chemical mechanisms involved with the creation of dimethyl ether according to one embodiment of the present invention.

FIG. 2 is a schematic diagram of a steady state isotope transient tracing of dimethyl ether using carbon 13 isotope tagging of carbon monoxide.

FIG. 3 is a block diagram identifying the compartmentalized model of the mechanism of conversion of synthesis gas over a Pd/AL2O3 catalyst using carbon 13 isotope tagging of carbon monoxide.

FIG. 4a is a graph showing the theoretically calculated Fractional Marking Level of dimethyl ether over time and the actual measured Fractional Marking Level of dimethyl ether over time.

FIG. 4b is a graph showing the simulated results of the Fractional Marking Level of ¹³CH₃OCH₃ and (¹³CH₃)₂O over time.

FIG. 5 is a graph showing the theoretically calculated Fractional Marking Level of methanol transient over time and the actual measured Fractional Marking Level of methanol transient over time.

FIG. 6 is a graph showing the theoretically calculated Fractional Marking Level of methane transient over time and the actual measured Fractional Marking Level of methane transient over time.

### DETAILED DESCRIPTION OF THE PRESENT INVENTION

It is known that dimethyl ether may be produced from gases such as carbon monoxide, carbon dioxide and hydrogen. These gases may be acquired by heating coal with high temperature steam, and are typically referred to as “syngases”.

In the past, syngases were introduced into a reaction chamber with a catalyst to produce various ratios of dimethyl ether, methanol and methane. Since the exact chemical mechanism and functioning of the catalysts were not previously understood, there would be varying amounts of these products produced.

Since the goal is to create a petroleum substitute which is economically feasible, the process should be optimized. In order to optimize the process one must be aware of the chemical interactions and chemical mechanisms involved.

Dr. Masood Otarod, the present inventor, has submitted the 2006 Otarod Paper as a Proposal to the National Science Foundation Sep. 15, 2006.

The attached 2006 Otarod Paper goes into detail explaining the steps involved with determining the chemical mechanism and functioning of the catalyst.

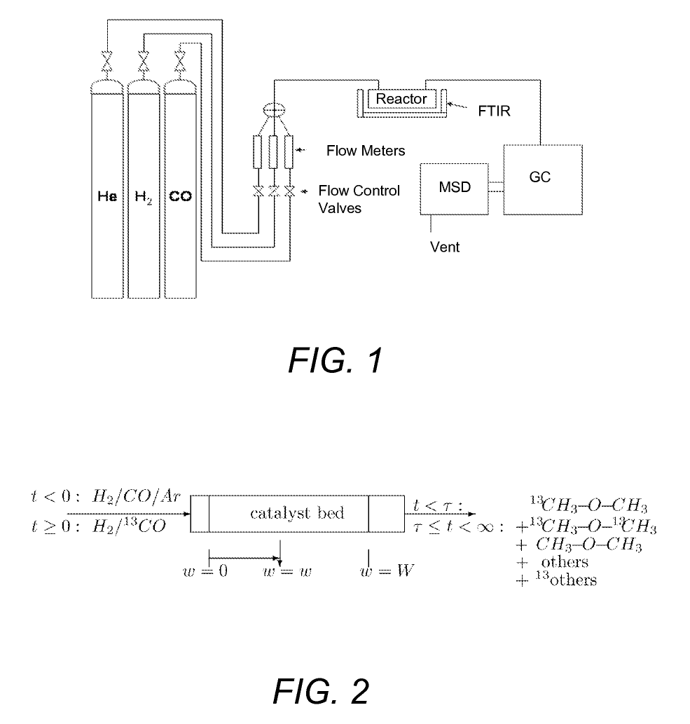

FIG. 1 is a flow system used in determining the chemical mechanisms involved with the creation of dimethyl ether according to one embodiment of the present invention.

Here the equipment is shown which is used in determining the constituents of the reaction. Helium, hydrogen and carbon monoxide are introduced at predefined rates into a catalyst filled reactor.

FIG. 2 is a schematic diagram of a steady state isotope transient tracing of dimethyl ether using carbon 13 isotope tagging of carbon monoxide. Here the input gases and the resulting reaction products are shown as they pass through a catalyst bed in the reactor.

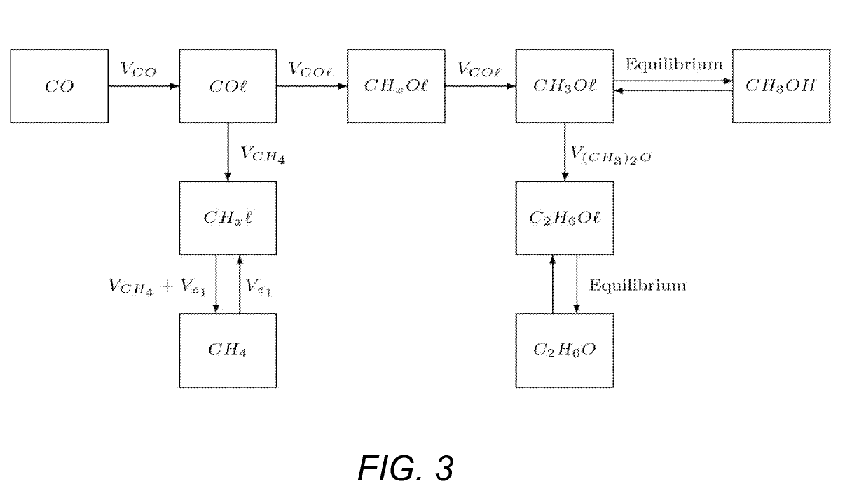

The 2006 Otarod Paper goes through a lengthy and detailed description of how the mechanism was determined and how the catalysts function. An overview of the reactions, intermediary reactions, chemical constituents and chemical mechanism are shown in the block diagram of FIG. 3.

FIG. 3 is a compartmentalized model of the mechanism of conversion of synthesis gas over a Pd/AL2O3 catalyst using carbon 13 isotope tagging of carbon monoxide.

Many of these reactions in the 2006 Otarod Paper are reversible and have a forward rate and a reverse rate. This means that reactants combine into products at a chemical rate defined as the forward rate. And products deteriorate into reactants at a chemical rate defined as the reverse rate. The reaction will favor one side of the equation under one set of conditions and the other for a different set of conditions.

These conditions include the concentration of the constituents which determined the rate at which the gases are introduced, the temperature which this occurs, the pressure under which the reactions take place, the presence of other contaminating constituents. The direction in which these reversible reactions will lean also includes the chemical composition, physical composition, surface area and other factors related to the catalysts used. Therefore, these can be adjusted to cause the reaction to produce the desired constituents.

However, since many of these reactions are occurring at the same time within the same reactor, changing a single condition, such as temperature, causes multiple reactions to change. Therefore, optimization includes correct selection of each of the multiple conditions which affect multiple reactions, to produce the optimum amount of dimethyl ether. Other constraints may be considered besides only optimization of the amount of dimethyl ether, such as minimization of hazardous byproducts, minimization of costs by reducing energy input, or minimization of costs by using less or less expensive raw materials.

The theoretical chemical mechanism proposed in the 2006 Otarod Paper fits well with the experimental results achieved.

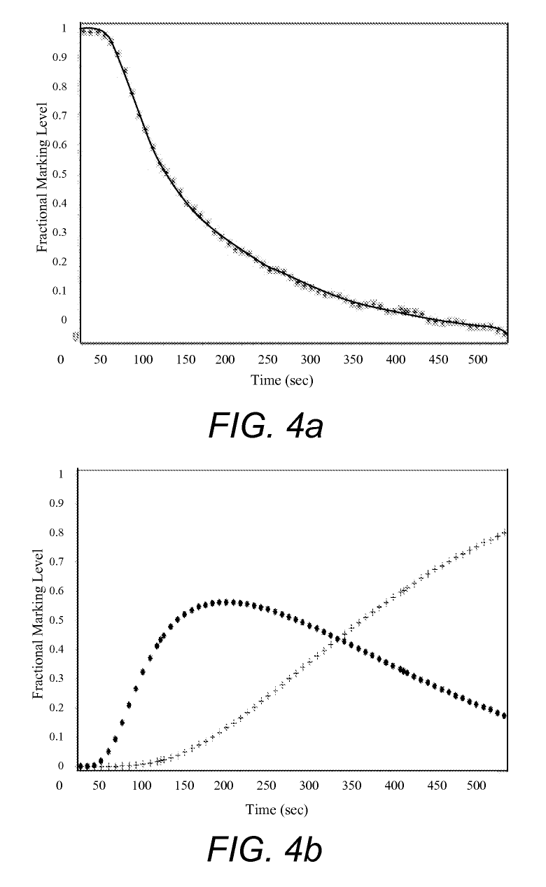

FIG. 4a is a graph showing the theoretically calculated Fractional Marking Level of dimethyl ether over time indicated by the symbols “-”, and the actual measured Fractional Marking Level of dimethyl ether over time indicated by the symbols “*”. These have remarkable correlation.

FIG. 4b is a graph showing the simulated results of the Fractional Marking Level of dimethyl ether over time indicated by the symbols “x”, and the simulated results of the Fractional Marking Level of the carbon 13 isotope of dimethyl ether over time indicated with solid circles.

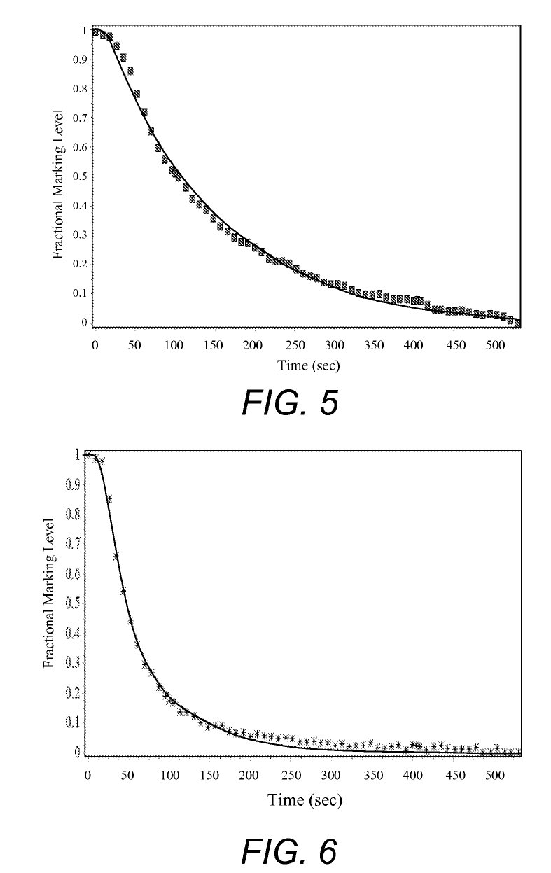

FIG. 5 is a graph showing the theoretically calculated Fractional Marking Level of methanol transient over time indicated by the symbols “-”, and the actual measured Fractional Marking Level of methanol transient over time indicated by the symbols “*”. Again, these graphs correlate very well.

FIG. 6 is a graph showing the theoretically calculated Fractional Marking Level of methane transient over time indicated by the symbols “-”, and the actual measured Fractional Marking Level of methane transient over time indicated by the symbols “*”. As above, these graphs correlate very well.

Now that the mechanism, the intermediate reactions, intermediate reactants, and intermediate products have been identified, the conditions may be adjusted to optimize the process towards the creation of dimethyl ether.

Therefore, optimum conditions as stated in the 2006 Otarod Paper, include introducing reactant gases according to the ratios defined by the chemical equations into a reactor. The reactor is filled with beads covered on the surface with a catalyst of which includes palladium and one of aluminum, copper, zinc, or mixtures of these. The catalyst is designed to have an acid side of the molecule. The acid side of the molecule functions to dehydrate methanol which attaches to it, reducing the concentration of this unwanted byproduct and increases the production of diethyl ether.

The reactor operates at a very high temperature on the order of 800-900 Deg. C.

The catalyst composition and use, along with other conditions are also disclosed in greater detail in the attached Paper.

While several presently preferred embodiments of the novel invention have been described in detail herein, many modifications and variations will now become apparent to those skilled in the art.

1. An improved method for creating dimethyl ether comprising the steps of:
a. providing a reactor is filled with beads covered on the surface with a catalyst molecules, at least some of the catalyst molecules having an acid side;
b. heating the reactor to approximately 800 degrees C.;
c. introducing reactant gases according to predefined ratios into a reactor wherein the acid side of the molecule functions to dehydrate methanol created from the reactant gases which attaches to it, reducing the concentration of this unwanted byproduct and increasing the production of diethyl ether.

2. The improved method for creating dimethyl ether of claim 1, wherein the catalyst further comprises aluminum.

3. The improved method for creating dimethyl ether of claim 1, wherein the catalyst further comprises copper.

4. The improved method for creating dimethyl ether of claim 1, wherein the catalyst further comprises zinc.

5. The improved method for creating dimethyl ether of claim 1, wherein one of the reactant gases is hydrogen.

6. The improved method for creating dimethyl ether of claim 1, wherein one of the reactant gases is carbon monoxide.

7. The improved method for creating dimethyl ether of claim 1, wherein one of the reactant gases is carbon dioxide.

8. The improved method for creating dimethyl ether of claim 1, wherein the reactor temperature is over 500 degrees C.

9. The improved method for creating dimethyl ether of claim 1, wherein the predefined volume ratio of hydrogen to carbon monoxide is 3 to 1.

10. The improved method for creating dimethyl ether of claim 1, wherein the predefined volume ratio of hydrogen to carbon dioxide is 3 to 1.
